(19) United States
(12) Patent Application Publication
(10) Pub. No.: US 2024/0257969 A1
(43) Pub. Date: Aug. 1, 2024

(54) Methods and Systems for Providing Clinical States of an Ocular Disease Based on Genomic Data and Phenotypic Data of Subjects

(71) Applicant: Avellino Lab USA, Inc., Menlo Park, CA (US)

(72) Inventors: Andrey Ptitsyn, St. Augustine, FL (US); Yelena Bykhovskaya, Los Angeles, CA (US); Genya Dana, Minneapolis, MN (US); Ranya Habash, Miami, FL (US)

(21) Appl. No.: 18/425,854

(22) Filed: Jan. 29, 2024

Related U.S. Application Data
(60) Provisional application No. 63/482,007, filed on Jan. 27, 2023.

(51) Int. Cl. G16H 50/20

(52) U.S. Cl. CPC G16H 50/20

(57) ABSTRACT

A method performed by a computer system including one or more electronic processors and memory includes receiving genomic data, ocular phenotypic data, and receiving ocular clinical diagnosis data for a plurality of subjects. The method also includes identifying a mapping function that maps the genomic data for the plurality of subjects to the ocular clinical diagnosis data for the plurality of subjects and a mapping function that maps the ocular phenotypic data for the plurality of subjects to the ocular clinical diagnosis data for the plurality of subjects. The method may also include obtaining one or more scores indicative of clinical states of an ocular disease for a respective subject of the plurality of subjects. The method may further include treating the plurality of subjects based on the one or more scores.

### RELATED APPLICATIONS

This application claims the benefit of, and priority to, U.S. Provisional Patent Application Ser. No. 63/482,007, filed Jan. 27, 2023, which is incorporated by reference herein in its entirety.

### TECHNICAL FIELD

This relates generally to methods and systems for diagnosis and/or prognosis of ocular diseases, and particularly to methods for diagnosis and/or prognosis of ocular diseases based on both genomic data and phenotypic data.

### BACKGROUND

Eyes are important organs, which play a critical role in human's visual perception. Ocular diseases are often diagnosed based on visually detectable changes to the eye (e.g., clouding of the cornea or lens). In some cases, analytical instruments, such as autorefractors, keratometers, and fundus cameras, are used to monitor changes to the eye. However, visually detectable changes provide only limited information associated with ocular diseases.

### SUMMARY

Accordingly, there is a need for methods and devices that can assist diagnosis and prognosis of ocular diseases that utilize information different from visually detectable changes. As described herein, methods and devices that utilize both phenotypic data and genomic data to provide scores indicative of clinical states of ocular diseases.

The above deficiencies and other problems associated with conventional methods are reduced or eliminated by methods and devices described herein. Such methods and devices can improve accuracy in diagnosis and/or prognosis of ocular diseases and may also enable and/or prognosis of ocular disease that were not possible using conventional methods (e.g., diagnosis of ocular diseases in an early stage before such ocular diseases can be detected by the conventional methods or prognosis of ocular diseases for changes that could not be detected by the conventional methods with acceptable certainty).

In accordance with some embodiments, a method performed by a computer system including one or more electronic processors and memory includes receiving genomic data for a plurality of subjects; receiving ocular phenotypic data for the plurality of subjects; receiving ocular clinical diagnosis data for the plurality of subjects; and identifying a first mapping function that maps the genomic data for the plurality of subjects to the ocular clinical diagnosis data for the plurality of subjects. The first mapping function maps genomic data for a respective person to a first score indicative of a clinical state of an ocular disease for the respective person. The method also includes identifying a second mapping function that maps the ocular phenotypic data for the plurality of subjects to the ocular clinical diagnosis data for the plurality of subjects. The second mapping function maps ocular phenotypic data for the respective person to the clinical state of the ocular disease for the respective person.

In accordance with some embodiments, a method performed by a computer system including one or more electronic processors and memory includes receiving genomic data for a plurality of subjects; receiving ocular phenotypic data for the plurality of subjects; receiving ocular clinical diagnosis data for the plurality of subjects; and identifying a mapping function that maps the genomic data for a respective subject of the plurality of subjects and the ocular phenotypic data for the respective subject of the plurality of subjects to the ocular clinical diagnosis data for the respective subject of the plurality of subjects. The mapping function maps genomic data for a respective person and ocular phenotypic data for the respective person to a score indicative of a clinical state of an ocular disease for the respective person.

In accordance with some embodiments, a method performed by a computer system including one or more electronic processors and memory includes receiving genomic data for a first person; receiving phenotypic data for the first person; obtaining a first score indicative of a first clinical state of an ocular disease for the first person by mapping the genomic data for the first person with a first mapping function that maps genomic data for a plurality of subjects to ocular clinical diagnosis data for the plurality of subjects; obtaining a second score indicative of a second clinical state of the ocular disease for the first person by mapping the phenotypic data for the first person with a second mapping function that is distinct from the first mapping function and maps phenotypic data for a plurality of subjects to clinical states of the ocular disease for the plurality of subjects; and providing the first score and the second score.

In accordance with some embodiments, a method performed by a computer system including one or more electronic processors and memory includes receiving genomic data for a first person; receiving phenotypic data for the first person; and obtaining a score indicative of a clinical state of an ocular disease for the first person by mapping the genomic data for the first person and the phenotypic data for the first person. The mapping function maps genomic data for a respective subject of a plurality of subjects and ocular phenotypic data for the respective subject of the plurality of subjects to a respective score indicative of a clinical state of the ocular disease for the respective subject of the plurality of subjects. The method also includes providing the score.

In accordance with some embodiments, a computer system includes one or more processors and memory one or more instructions, which, when executed by the one or more processors, cause the one or more processors to perform any method described herein.

In accordance with some embodiments, a computer readable storage medium stores one or more programs for execution by one or more processors, the one or more programs including instructions for performing any method described herein.

Thus, the disclosed methods and devices improves diagnosis and/or prognosis of an ocular disease.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1 is a schematic diagram showing a system for vision characterization in accordance with some embodiments.

FIG. 2 is a block diagram illustrating electronic components of an optical device in accordance with some embodiments.

FIG. 3 is a block diagram illustrating combination of image or topographic data in accordance with some embodiments.

FIG. 4 illustrates combination of multi-modal data in accordance with some embodiments.

FIG. 5 is a flow diagram illustrating a method of utilizing topographic data and genetic data in accordance with some embodiments.

FIG. 6A shows a graphical presentation of two scores in accordance with some embodiments.

FIG. 6B shows a graphical presentation of combined scores in accordance with some embodiments.

FIG. 7 is a flow diagram illustrating a method of identifying mapping functions in accordance with some embodiments.

FIG. 8 is a flow diagram illustrating a method of identifying a mapping function that utilizes both genomic data and phenotypic data in accordance with some embodiments.

FIG. 9 is a flow diagram illustrating a method of obtaining mapping scores in accordance with some embodiments.

FIG. 10 is a flow diagram illustrating a method of obtaining a mapping score based on both genomic data and phenotypic data in accordance with some embodiments.

These figures are not drawn to scale unless indicated otherwise.

### DETAILED DESCRIPTION

As explained above, visually detectable changes in phenotypic data provide only limited information associated with ocular diseases. In addition, the use of the genetic data alone does not consider the current state of ocular diseases in subjects. The methods and devices described herein utilize both the genetic data and the phenotypic data to mitigate shortfalls and augment advantages of both phenotypic data (e.g., corneal scan data) and genetic data.

Reference will be made to embodiments, examples of which are illustrated in the accompanying drawings. In the following description, numerous specific details are set forth in order to provide a thorough understanding of the various described embodiments. However, it will be apparent to one of ordinary skill in the art that the various described embodiments may be practiced without these particular details. In other instances, methods, procedures, components, circuits, and networks that are well-known to those of ordinary skill in the art are not described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first image sensor could be termed a second image sensor, and, similarly, a second image sensor could be termed a first image sensor, without departing from the scope of the various described embodiments. The first image sensor and the second image sensor are both image sensors, but they are not the same image sensor.

The terminology used in the description of the embodiments herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

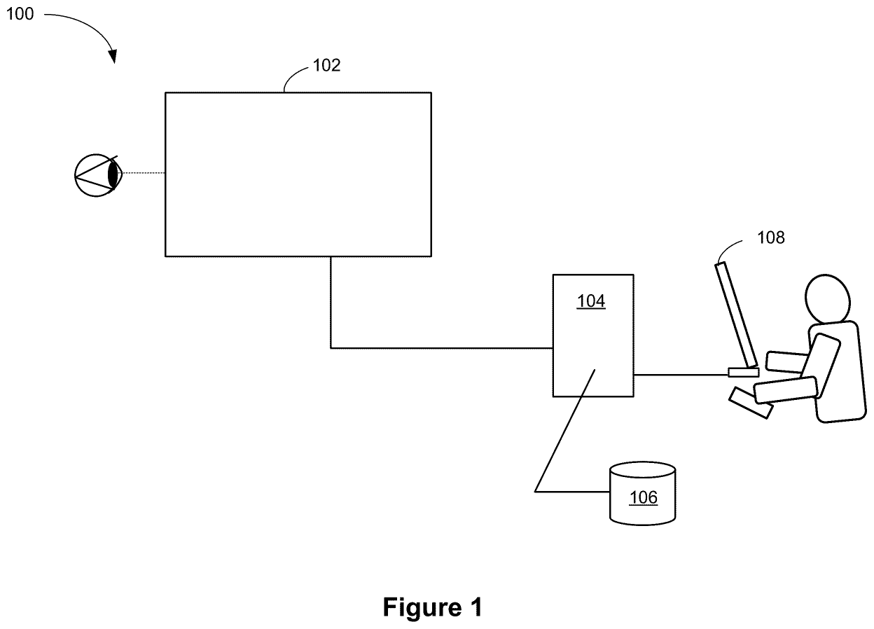

FIG. 1 is a schematic diagram showing a system 100 for vision characterization in accordance with some embodiments. The system 100 includes a measurement device 102, a computer system 104, a database 106, and a display device 108. The measurement device 102 performs a vision characterization of an eye of a patient (e.g., using light source and an imaging sensor) and provides imaging results and vision profile metrics of the characterized eye. In some cases, the measurement device 102 includes a topography measurement device. For example, the measurement device 102 may project a predefined light pattern (e.g., a Placido disk pattern including a plurality of concentric rings) and detect an image of reflected light. Based on the distortion of the concentric rings in the detected image, a topography of a corneal surface may be determined. In some cases, the measurement device 102 includes a wavefront measurement device, such as a Shack-Hartmann wavefront sensor, that is configured to perform wavefront measurements. The display device 108 shows the imaging results and vision profile metrics acquired by the measurement device 102. The database 106 stores imaging results and vision profile metrics acquired by the measurement device 102. As described herein, in response to receiving the results from the measurement device 102, the system 100 may also provide one or more scores indicative of a clinical state of an ocular disease.

The computer system 104 may include one or more computers or central processing units (CPUs). The computer system 104 is in communication with each of the measurement device 102, the database 106, and the display device 108.

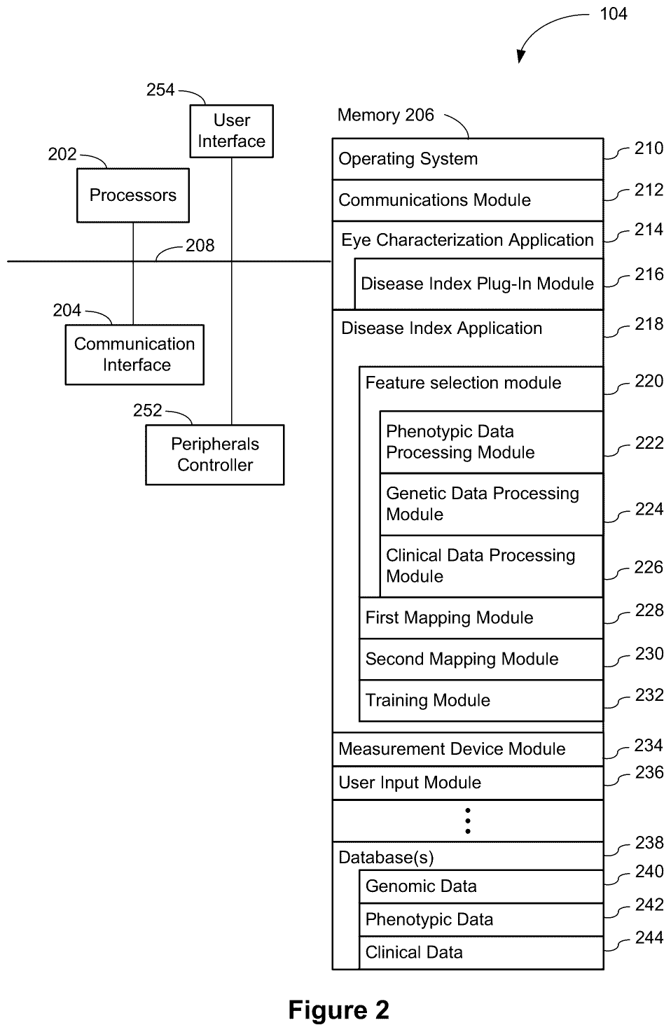

FIG. 2 shows block diagram illustrating electronic components of computer system 104 in accordance with some embodiments. Computer system 104 includes one or more processors or processing units 202 (central processing units, application processing units, application-specific integrated circuit, etc., which are also called herein processors), one or more network or other communications interfaces 204, memory 206, and one or more communication buses 208 for interconnecting these components. In some embodiments, communication buses 208 include circuitry (sometimes called a chipset) that interconnects and controls communications between system components. In some embodiments, system 100 includes a user interface 254 (e.g., a user interface having the display device 108, which can be used for displaying acquired images, one or more buttons, and/or other input devices). In some embodiments, computer system 104 also includes peripherals controller 252, which is configured to control operations of components of the measurement device 102, such as a light source and an image sensor (e.g., initiating the light source to emit light, and/or receiving information, such as images, from the image sensor).

In some embodiments, communications interfaces 204 include wired communications interfaces and/or wireless communications interfaces (e.g., Wi-Fi, Bluetooth, etc.).

Memory 206 of computer system 104 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 206 may optionally include one or more storage devices remotely located from the processors 202. Memory 206, or alternately the non-volatile memory device(s) within memory 206, comprises a computer readable storage medium (which includes a non-transitory computer readable storage medium and/or a transitory computer readable storage medium). In some embodiments, memory 206 includes a removable storage device (e.g., Secure Digital memory card, Universal Serial Bus memory device, etc.). In some embodiments, memory 206 or the computer readable storage medium of memory 206 stores the following programs, modules and data structures, or a subset thereof:
operating system 210 that includes procedures for handling various basic system services and for performing hardware dependent tasks;
network communication module (or instructions) 212 that is used for connecting computer system 104 to other computers (e.g., clients and/or servers) via one or more communications interfaces 204 and one or more communications networks, such as the Internet, other wide area networks, local area networks, metropolitan area networks, and so on;
eye characterization application 214 (which may be a stand-alone application or an application that runs in a web browser) that characterizes an eye, such as by providing a refractive power of the eye and/or providing a topography (e.g., corneal topography) of the eye;
disease index application 218 that provides one or more scores (e.g., disease indexes) indicative of an ocular disease;
measurement device module 234 that controls operations of one or more light sources and one or more image sensors in the measurement device 102 (e.g., for receiving images from the measurement device 102);
user input module 236 configured for handling user inputs on computer system 104 (e.g., pressing of buttons on computer system 104 or pressing of buttons on a user interface, such as a keyboard, mouse, or touch-sensitive display, that is in communication with computer system 104); and
one or more databases 238 (e.g., database 106) that store information acquired by the measurement device 102.

In some embodiments, memory 206 also includes one or both of:
user information (e.g., information necessary for authenticating a user of computer system 104); and
patient information (e.g., optical measurement results and/or information that can identify patients whose optical measurement results are stored in the one or more databases 238 on computer system 104).

In some embodiments, eye characterization application 214 includes disease index plug-in module 216 (instead of, or in addition to, disease index application 218) for providing one or more scores indicative of an ocular disease.

In some embodiments, disease index application 218 includes the following programs, modules and data structures, or a subset or superset thereof:
feature selection module 220 configured for identifying (e.g., automatically identifying) one or more reference markings in an image captured (e.g., recorded, acquired) by the measurement device 102, which may include one or more of the following:
   periphery reference marking identification module 222 configured for identifying (e.g., automatically identifying) one or more periphery reference markings in an image captured (e.g., recorded, acquired) by the measurement device 102;
   angular reference marking identification module 224 configured for identifying (e.g., automatically identifying) one or more angular reference markings in an image captured (e.g., recorded, acquired) by the measurement device 102; and
   illumination marking identification module 226 configured for identifying (e.g., automatically identifying) one or more illumination markings in an image captured (e.g., recorded, acquired) by the measurement device 102;
first mapping module 228 configured for mapping genetic data (e.g., genomic data 240) to clinical data or clinical states of subjects;
second mapping module 230 configured for mapping phenotypic data (e.g., image or topographic information for an eye) to clinical data or clinical states of subjects; and
training module 232 configured for identifying one or more mapping functions based on training data set (e.g., clinical data and at least one of: genomic data or phenotypic data for a plurality of subjects).

In some embodiments, measurement device module 234 includes the following programs and modules, or a subset or superset thereof:
a light source module configured for initiating a light source (through peripherals controller 252) to emit light;
an image sensing module configured for receiving images from an image sensor;
an image acquisition module configured for capturing one or more images of a patient's eye(s) using the measurement device 102; and
an image stabilization module configured for reducing blurring during acquisition of images by image sensors.

In some embodiments, the computer system 104 may include other modules such as:
a presentation module configured for presenting measurement and analysis results from second analysis module (e.g., graphically displaying images received from an image sensor, presenting cornea curvatures determined from images received from the image sensor, sending the results to another computer, etc.); and
a light pattern analysis module configured for analyzing a projected pattern of light (e.g., measuring displacements and/or distortion of concentric rings in the collected image).

In some embodiments, the one or more databases 238 may store any of: phenotypic data 242 (e.g., images or topographic information) for subjects and/or a subset thereof (e.g., selected features from the images or topographic information), genetic data (e.g., genomic data) 240 for the subjects, clinical data 244 for the subjects, mapping functions, or mapped scores.

Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. In some embodiments, memory 206 may store a subset of the modules and data structures identified above. Furthermore, memory 206 may store additional modules and data structures not described above.

Notwithstanding the discrete blocks in FIG. 2, these figures are intended to be a functional description of some embodiments, although, in some embodiments, the discrete blocks in FIG. 2 can be a structural description of functional elements in the embodiments. One of ordinary skill in the art will recognize that an actual implementation might have the functional elements grouped or split among various components. In practice, and as recognized by those of ordinary skill in the art, items shown separately could be combined and some items could be separated. For example, in some embodiments, measurement device module 234 is part of disease index application 218. In other embodiments, feature selection module 220, first mapping module 228, second mapping module 230, and training module 232 are implemented as separate applications. In some embodiments, one or more programs, modules, or instructions may be implemented in measurement device 102 instead of computer system 104. In some embodiments, training module 232 is implemented in a computer system that is separate from a computer system that includes first mapping module 228 and second mapping module 230.

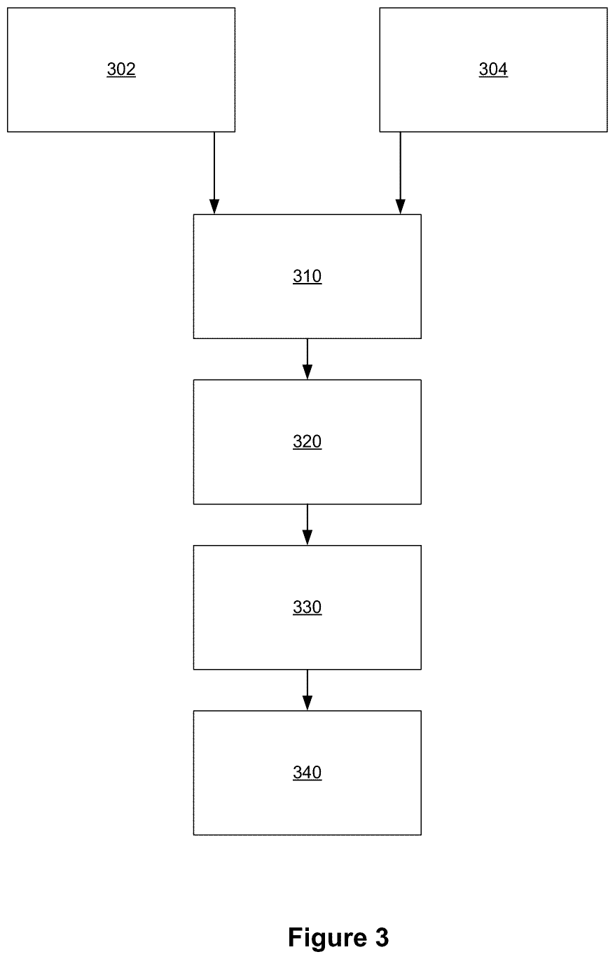

FIG. 3 is a block diagram illustrating combination of image or topographic data in accordance with some embodiments.

In some embodiments, at least a subset 302 of phenotypic data (e.g., topographic data), which has been previously collected and stored, is used for identifying one or more mapping functions.

In some embodiments, at least a subset 304 of phenotypic data (e.g., topographic data), which has been collected by a measurement device (e.g., the measurement device 102), is used for identifying one or more mapping functions.

In some embodiments, both the subset 302 and the subset 304 of phenotypic data are used for identifying one or more mapping functions. For this, a combination of the subset 302 and the subset 304 is prepared (310).

In some embodiments, the combination of the subset 302 and the subset 304 is normalized and/or scaled (320).

In some embodiments, a subset of the combination (e.g., features) is selected (330). For example, a portion of an image with high relevance to an ocular disease (e.g., an image of a particular region of an eye that is related to the ocular disease) or a curvature of a particular portion of the eye with high relevance to the ocular disease is selected.

In some embodiments, the subset of the combination is provided (340) for identifying one or more mapping functions.

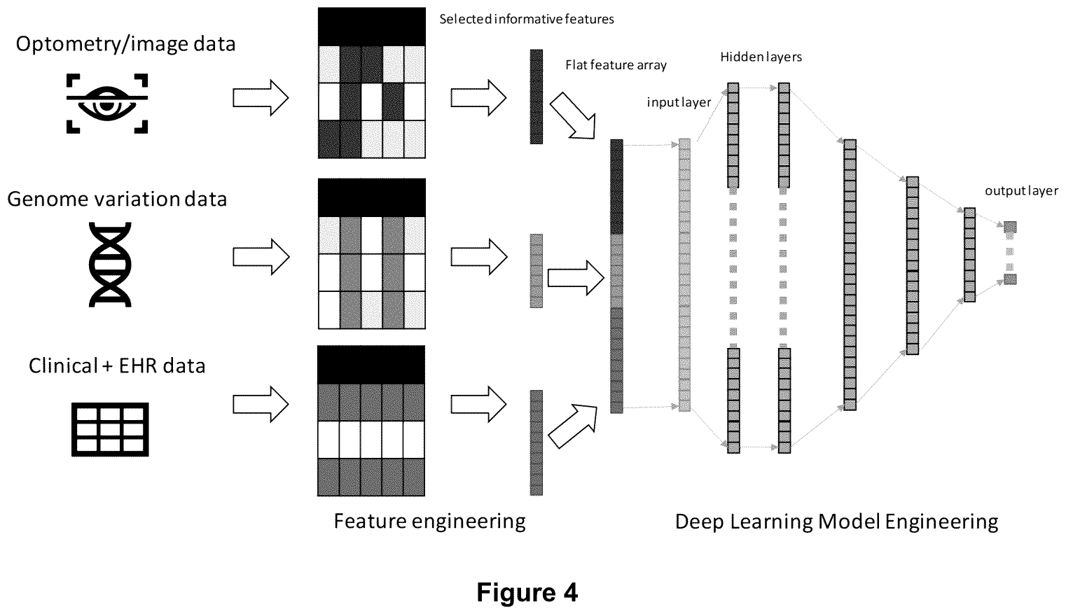

FIG. 4 illustrates combination of multi-modal data in accordance with some embodiments.

As described with respect to FIG. 3, in some embodiments, a subset of the phenotypic data (e.g., corneal topographic data) is used for identifying one or more mapping functions. Similarly, in some embodiments, a subset of the genetic data (e.g., genomic data) is used for identifying one or more mapping functions. For example, instead of utilizing a whole genome sequence, information identifying particular mutations in the genome may be used for identifying one or more mapping functions. Furthermore, in some embodiments, a subset of the clinical data is used for identifying one or more mapping functions. For example, a clinical diagnosis of a particular ocular disease may be selected and used for identifying one or more mapping functions.

As shown in FIG. 4, in some embodiments, the phenotypic data, the genetic data, and/or the clinical data (e.g., electronic health records, history of health and disease states, socio-economic profile, etc.) may be flattened prior to the selection of a respective subset. In some embodiments, the phenotypic data, the genetic data, and/or the clinical data may be flattened in conjunction with, or subsequent to, the selection of a respective subset.

In some embodiments, the subsets of data are combined (e.g., concatenated).

In some embodiments, the combined subsets are used to identify one or more mapping functions (e.g., a mapping function that maps the subset of phenotypic data and the subset of genetic data to a score indicative of a clinical state for a particular ocular disease). In some embodiments, the one or more mapping functions are identified or obtained through machine learning (e.g., using deep neural networks, deep belief networks, deep reinforcement learning, recurrent neural networks, convolutional neural networks, etc.). For example, the combined data (e.g., the combined subsets) allows the use of a single neural network to identify the one or more mapping functions, instead of using separate neural networks (e.g., using a first neural network for mapping the phenotypic data to a score indicative of a clinical state and using a separate, second neural network for mapping the genomic data to a score indicative of the clinical state).

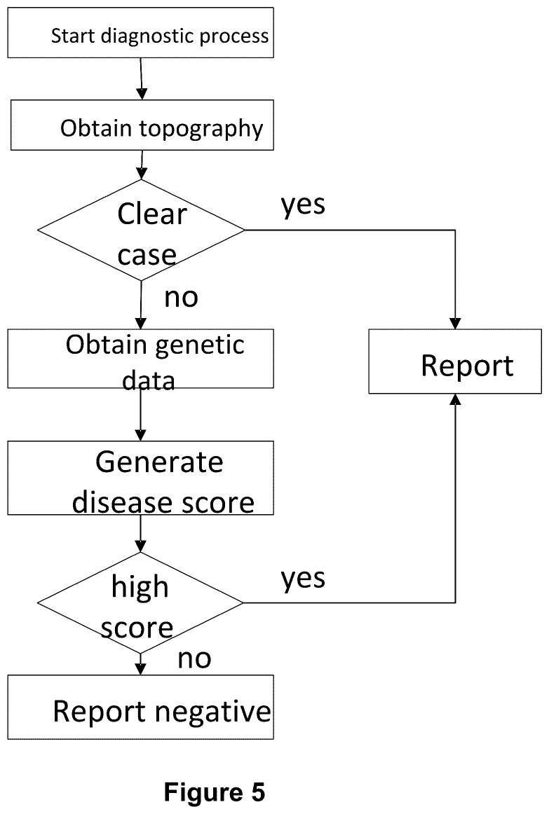

FIG. 5 is a flow diagram illustrating a method of utilizing topographic data and genetic data in accordance with some embodiments.

In some embodiments, a diagnosis process starts with topography data for a particular subject.

In some embodiments, in accordance with a determination (e.g., by a computer system) that a confidence in calling out the presence of a particular ocular disease (e.g., keratoconus or glaucoma) is above a predefined threshold, the computer system provides a report that the particular ocular disease is present in the subject.

In some embodiments, in accordance with a determination that a confidence in calling out the presence of the particular ocular disease is below the predefined threshold, the computer system obtains genetic data of the subject.

Thereafter, the computer system generates a score based on the phenotypical data (e.g., corneal topography) for the particular subject and the genetic data for the particular subject, where the score indicates a clinical state of the particular ocular disease in the subject.

In some embodiments, in accordance with a determination that the score is above a second predefined threshold, the computer system provides a report that the particular ocular disease is likely to be present in the subject.

In some embodiments, in accordance with a determination that the score is below the second predefined threshold, the computer system reports that the particular ocular disease is not likely to be present in the subject.

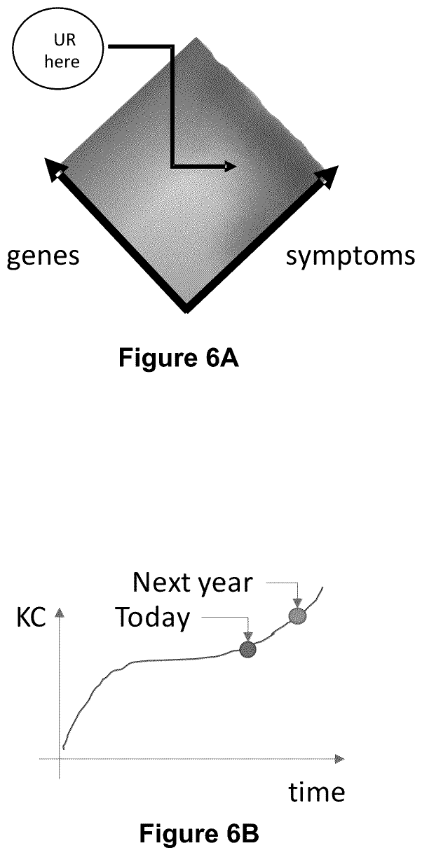

FIG. 6A shows a graphical presentation of two scores in accordance with some embodiments.

In some embodiments, two or more scores are obtained from the phenotypic data and the genetic data. For example, in some embodiments, a first score is obtained from the genetic data without using the phenotypic data, and a second score is obtained from the phenotypic data without using the genetic data. A combination of the first score and the second score may be provided to indicate a clinical state of an ocular disease in a subject. In some embodiments, as shown in FIG. 6A, the combination of the first score and the second score may be graphically presented in a chart, where the first score represents a coordinate on a first axis (e.g., the “genes” axis in FIG. 6A) and the second score represents a coordinate on a second axis (e.g., the “symptoms” axis in FIG. 6A). In some embodiments, the two or more scores are produced together in a single computation.

FIG. 6B shows a graphical presentation of combined scores in accordance with some embodiments.

Instead of presenting a pair of scores for a subject, a single score that is based on both the phenotypic data and the genetic data may be used. In some embodiments, a series of scores determined over a period of time may be used to predict the score in the future (e.g., indicating progression of the ocular disease over time), which may be graphically presented as shown in FIG. 6B.

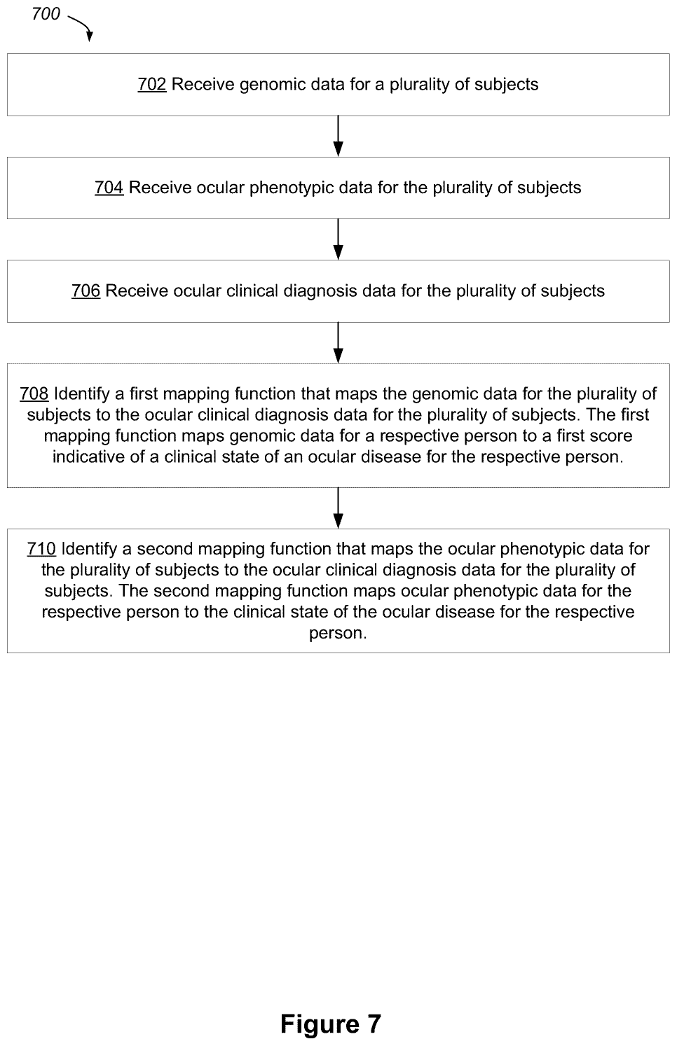

FIG. 7 is a flow diagram illustrating a method 700 of identifying mapping functions in accordance with some embodiments. In some embodiments, the method 700 is performed by a computer system (e.g., computer system 104) including one or more electronic processors and memory.

The method 700 includes (702) receiving genomic data for a plurality of subjects.

The method 700 includes (704) receiving ocular phenotypic data for the plurality of subjects.

The method 700 includes (706) receiving ocular clinical diagnosis data for the plurality of subjects.

The method 700 includes (708) identifying a first mapping function that maps the genomic data for the plurality of subjects to the ocular clinical diagnosis data for the plurality of subjects. The first mapping function maps genomic data for a respective person to a first score indicative of a clinical state of an ocular disease for the respective person.

The method 700 includes (710) identifying a second mapping function that maps the ocular phenotypic data for the plurality of subjects to the ocular clinical diagnosis data for the plurality of subjects, wherein the second mapping function maps ocular phenotypic data for the respective person to the clinical state of the ocular disease for the respective person.

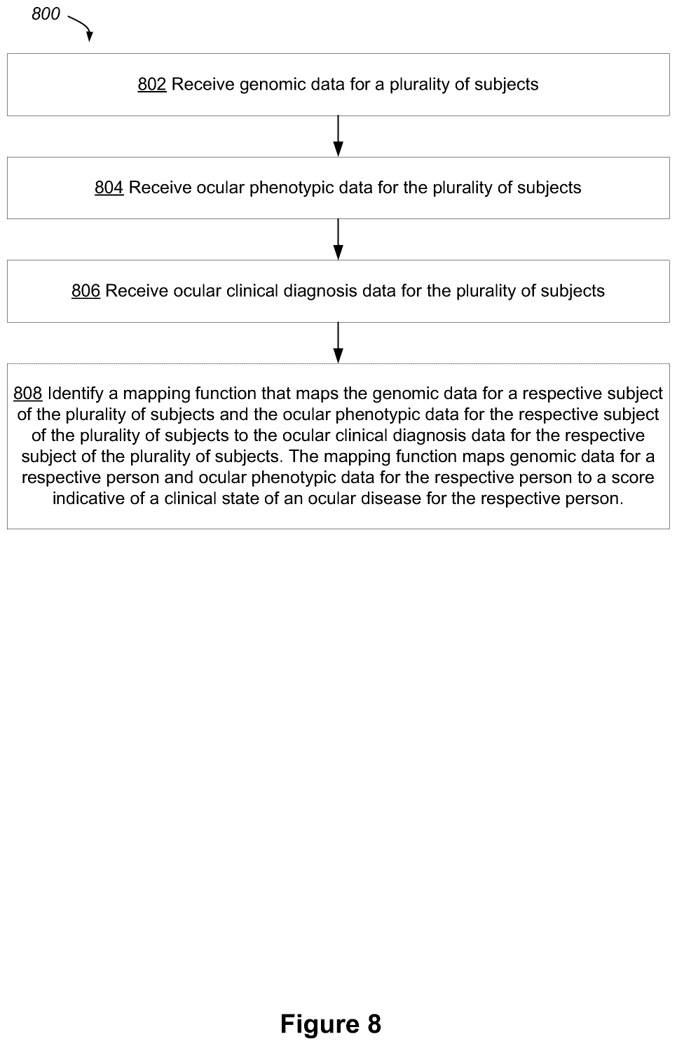

FIG. 8 is a flow diagram illustrating a method 800 of identifying a mapping function that utilizes both genomic data and phenotypic data in accordance with some embodiments. In some embodiments, the method 800 is performed by a computer system (e.g., computer system 104) including one or more electronic processors and memory.

The method 800 includes (802) receiving genomic data for a plurality of subjects.

The method 800 includes (804) receiving ocular phenotypic data for the plurality of subjects.

The method 800 includes (806) receiving ocular clinical diagnosis data for the plurality of subjects.

The method 800 includes (808) identifying a mapping function that maps the genomic data for a respective subject of the plurality of subjects and the ocular phenotypic data for the respective subject of the plurality of subjects to the ocular clinical diagnosis data for the respective subject of the plurality of subjects. The mapping function maps genomic data for a respective person and ocular phenotypic data for the respective person to a score indicative of a clinical state of an ocular disease for the respective person.

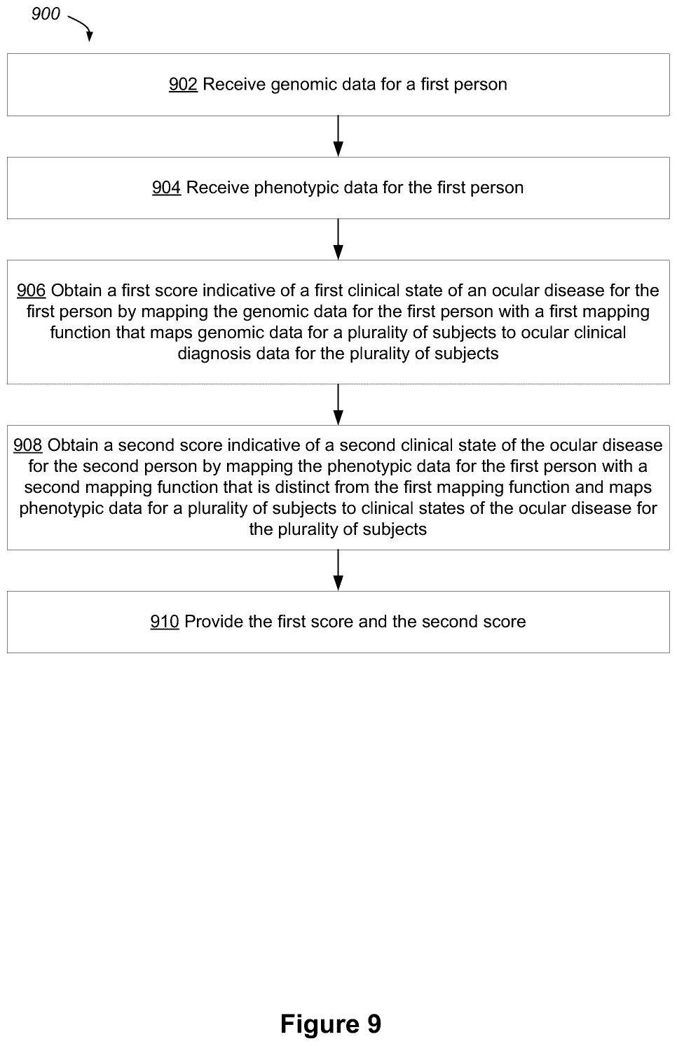

FIG. 9 is a flow diagram illustrating a method 900 of obtaining mapping scores in accordance with some embodiments. In some embodiments, the method 900 is performed by a computer system (e.g., computer system 104) including one or more electronic processors and memory.

The method 900 includes (902) receiving genomic data for a first person.

The method 900 includes (904) receiving phenotypic data for the first person.

The method 900 includes (906) obtaining a first score indicative of a first clinical state of an ocular disease for the first person by mapping the genomic data for the first person with a first mapping function that maps genomic data for a plurality of subjects to ocular clinical diagnosis data for the plurality of subjects.

The method 900 includes (908) obtaining a second score indicative of a second clinical state of the ocular disease for the first person by mapping the phenotypic data for the first person with a second mapping function that is distinct from the first mapping function and maps phenotypic data for a plurality of subjects to clinical states of the ocular disease for the plurality of subjects.

The method 900 includes (910) providing the first score and the second score.

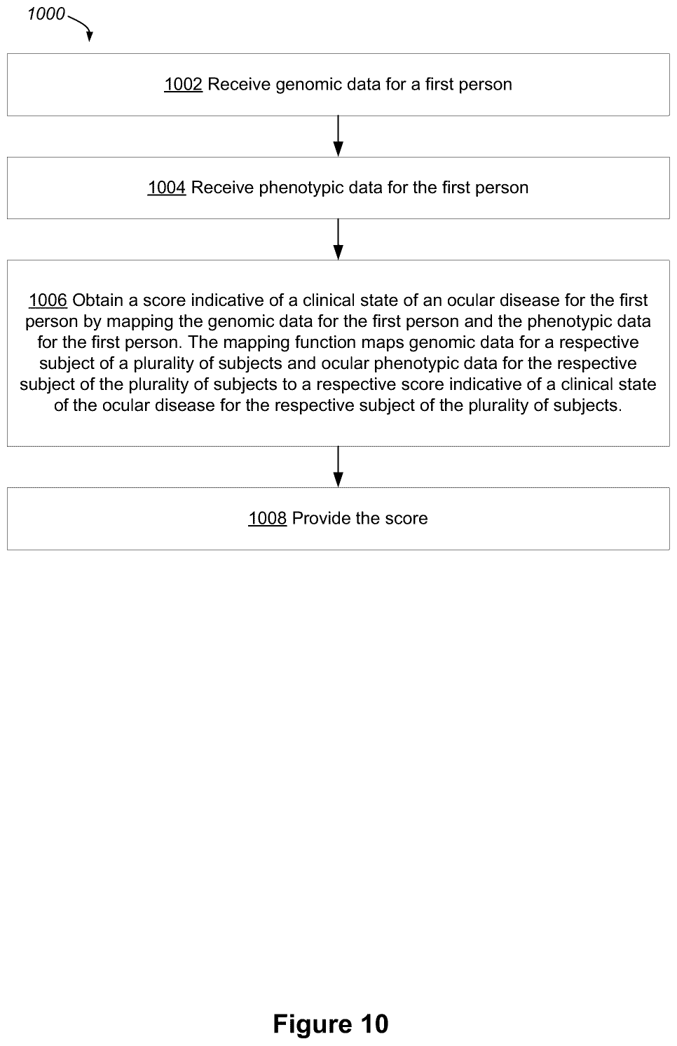

FIG. 10 is a flow diagram illustrating a method 1000 of obtaining a mapping score based on both genomic data and phenotypic data in accordance with some embodiments. In some embodiments, the method 1000 is performed by a computer system (e.g., computer system 104) including one or more electronic processors and memory.

The method 1000 includes (1002) receiving genomic data for a first person.

The method 1000 includes (1004) receiving phenotypic data for the first person.

The method 1000 includes (1006) obtaining a score indicative of a clinical state of an ocular disease for the first person by mapping the genomic data for the first person and the phenotypic data for the first person. The mapping function maps genomic data for a respective subject of a plurality of subjects and ocular phenotypic data for the respective subject of the plurality of subjects to a respective score indicative of a clinical state of the ocular disease for the respective subject of the plurality of subjects.

The method 1000 includes (1008) providing the score.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the invention and the various described embodiments with various modifications as are suited to the particular use contemplated.

What is claimed is:

1. A method performed by a computer system including one or more electronic processors and memory, the method comprising:
receiving genomic data for a plurality of subjects;
receiving ocular phenotypic data for the plurality of subjects;
receiving ocular clinical diagnosis data for the plurality of subjects;
identifying a first mapping function that maps the genomic data for the plurality of subjects to the ocular clinical diagnosis data for the plurality of subjects, wherein the first mapping function maps genomic data for a respective person to a first score indicative of a clinical state of an ocular disease for the respective person; and
identifying a second mapping function that maps the ocular phenotypic data for the plurality of subjects to the ocular clinical diagnosis data for the plurality of subjects, wherein the second mapping function maps ocular phenotypic data for the respective person to the clinical state of the ocular disease for the respective person.

2. The method of claim 1, further comprising:
storing the first mapping function and the second mapping function in the memory.

3. The method of claim 1, further comprising:
providing the first mapping function and the second mapping function to a second computer system that is separate from the computer system.

4. The method of claim 1, wherein:
the ocular disease is keratoconus.

5. The method of claim 1, wherein:
the ocular disease is glaucoma.

6. The method of claim 1, wherein:
the ocular phenotypic data includes ocular topography information.

7. The method of claim 1, further comprising:
selecting a subset, less than all, of the genomic data for the plurality of subjects for identifying the first mapping function.

8. The method of claim 1, further comprising:
selecting a subset, less than all, of the ocular phenotypic data for the plurality of subjects for identifying the second mapping function.

9. The method of claim 1, further comprising:
selecting a subset, less than all, of the ocular clinical diagnosis data for the plurality of subjects for at least one of: identifying the first mapping function or identifying the second mapping function.

10. The method of claim 1, wherein:
at least one of the first mapping function or the second mapping function is identified by using a deep learning model.

11. A computer system, comprising:
one or more processors; and
memory storing instructions, which, when executed by the one or more processors, cause the one or more processors to perform the method of claim 1.

12. A non-transitory computer readable storage medium storing one or more programs for execution by one or more processors, the one or more programs including instructions for performing the method of claim 1.
